(19) United States
(12) Patent Application Publication
(10) Pub. No.: US 2005/0106193 A1
(43) Pub. Date: May 19, 2005

(54) Ethoxylated polyurethane viscosity enhancers

(76) Inventors: Albert A. Zofchak, Holmdel, NJ (US); John Carson, Union City, NJ (US); Jebadiah Jorden, Matawan, NJ (US)

Correspondence Address: Henry D. Coleman;COLEMAN SUDOL SAPONE, P.C., 714 Colorado Avenue, Bridgeport, CT, 06605-1601, US

(21) Appl. No.: 10/714,772

(22) Filed: Nov. 17, 2003

(51) Int. Cl. A61K 7/06; A61K 7/075; A61K 7/08

(52) U.S. Cl. 424/401; 424/70.31

(57) ABSTRACT

The invention provides personal care products comprising fatty ethoxylated dimeric urethane compounds that enhance the viscosity of constituent water based surfactant systems. In compositions and methods of the invention, ethoxylated nonionic, urethane dimers increase the viscosity of water based surfactant systems and serve as thickeners. These thickeners produce clear solutions in combination with surfactants and can be made sufficiently water soluble to produce clear solutions in water. The thickeners are compatible with anionic, cationic, amphoteric and nonionic surfactants and are functional and stable over a wide pH range.

### FIELD OF THE INVENTION

The invention provides personal care products comprising fatty ethoxylated dimeric urethane compounds that enhance the viscosity of constituent water based surfactant systems. In compositions and methods of the invention, ethoxylated nonionic, urethane dimers increase the viscosity of personal care product water based surfactant systems and serve as thickeners. These thickeners produce clear solutions in combination with surfactants and can be made sufficiently water soluble to produce clear solutions in water. The thickeners are compatible with anionic, cationic, amphoteric and nonionic surfactants and are functional and stable over a wide pH range.

### BACKGROUND OF THE INVENTION

Viscosity control is necessary in personal care products to ensure stability, consistent dispensing, and desirable aesthetic properties. Personal care product viscosity can be controlled in many different ways. One common method uses water soluble polymers such as cellulosics, acrylates, natural gums, ethoxylates and related derivatives.

Ethoxylate thickeners typically comprise fatty acid esters of highly ethoxylated diols and polyols and include PEG 6000 distearate, a 150 mole ethoxylate formed by reaction of one mole of ethylene glycol, 150 moles of ethylene oxide and two moles of stearic acid. PEG 6000 distearate has been used as a viscosity building agent in a number of baby shampoos and has represented the standard in the industry. A 5% w/w concentration of PEG 6000 in water has a viscosity of about 1500 to 2000 cps. Such a solution in water becomes cloudy/pearlescent over time and viscosity is reasonably stable at neutral pH. PEG 6000 distearate forms a clear solution at 3% w/w concentration in 3% active sodium laurylether (2) sulfate (SLES2) and such a solution has a viscosity of about 25,000 cps. By way of comparison, water has a viscosity of about 1 to 2 cps and a 3% SLES2 solution also has a viscosity that is less than 5 cps.

Glucamate DOE 120 (Dow Chemical) can be used as a personal care product thickener. Glucamate DOE 120 comprises about 120 moles of ethoxylate formed by the esterification of one mole of (120 mole ethoxylated) methyl glucoside with two moles of oleic acid. When solubilized in water at a concentration of 5% w/w, a clear solution is formed and the solution has a viscosity of less than 50 cps. A 3% w/w solution of Glucamate DOE 120 in 3% active SLES2 yields a clear solution that has a viscosity of less than 50 cps.

Crothix (Croda Inc.) can be used as a personal care product thickener and is a tetra stearate ester made from a one hundred fifty mole ethoxylate of pentaerythritol. This product is not readily water soluble and, when mixed with water at 5% w/w, produces a cloudy/opaque discontinuous mixture containing numerous hydrated, sticky gel particles. Upon standing, this mixture becomes discontinuous with a hazy, low viscosity, watery layer at the top of the container and a cloudy/opaque soft, sticky gel layer on the bottom. No viscosity measurement of this mixture could be made. A 3% w/w solution in 3% active SLES2 produces a clear solution with a viscosity of about 1400 cps.

A typical baby shampoo formulation may include, for example, a combination of ethoxylated sorbitan ester, amphoteric surfactant, anionic surfactant and PEG 6000 distearate to yield a baby shampoo which is non-irritating to children's eyes (essentially a No Tear Shampoo).

All of the aforementioned thickeners are esters and, as such, have limited stability in water due to hydrolysis of the ester group. When the ester is hydrolyzed, viscosity decreases and clarity diminishes. Hydrolysis is problematic if product pH is either extremely acidic or basic. Limited hydrolytic stability limits the use of ester type viscosity builders and such compositions cannot be used in certain personal care products.

Foaming is also a problem in formulations that contain sorbitan ester ethoxylate, PEG 6000 distearate, or amphoterics as thickeners. PEG 6000 distearate, when used in concentrations approaching about 1.5% by weight, can decrease the foam height as measured by Ross-Miles foam determination equipment.

The need continues to exist, therefore, for thickeners that are useful in personal care products such as shampoos, that enhance the viscosity of constituent water based surfactant systems, and that are sufficiently water soluble to produce clear solutions in water. In particular, the need exists for personal care product thickeners that solubilize in the product and result in product viscosities preferably in the range of from about from about 5,000 to about 150,000 cps. Such thickeners should not depress foaming, should be stable over a wide pH range, and should be compatible with a broad range of surfactants.

### SUMMARY OF THE INVENTION

The invention provides personal care products comprising fatty ethoxylated dimeric urethane compounds that enhance the viscosity of constituent water based surfactant systems. In compositions and methods of the invention ethoxylated nonionic urethane dimers increase the viscosity of water based surfactant systems and serve as thickeners. These thickeners produce clear solutions in combination with surfactants and can be made sufficiently water soluble to produce clear solutions in water. While paint thickeners based on ethoxylated urethanes are known, the invention provides novel compositions and methods that use ethoxylated nonionic urethane dimers as thickeners in a broad array of personal care product applications.

The thickeners employed in the instant invention are compatible with anionic, cationic, amphoteric and nonionic surfactants and are functional and stable over a wide pH range. Thickeners used in the invention are long chain alcohol ethoxylated compounds in which the total ethoxylation ranges from about 100 to 240 moles of ethylene oxide, with a preferred amount of ethoxylation of approximately 130-140 moles, most preferably about 140 moles.

In particular in preferred aspects, the invention provides personal care products comprising fatty ethoxylated dimeric urethane compounds of the formula (I): 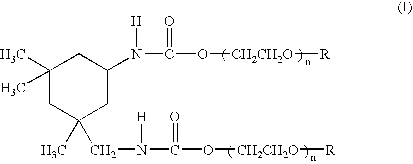
where n is a whole number from 50 to 120, preferably about 70 to 100, more preferably about 65-75, and most preferably about 70; and
R is a C₁₂-C₂₄ (preferably, C₁₅-C₂₄) alkyl or alkenyl group.

Compounds of formula (I) can be made by reacting two moles of an ethoxylated fatty alcohol with a diisocyanate, most preferably isophorone diisocyanate.

When incorporated into personal care products such as lotions and shampoo formulations in accordance with the invention, compounds of formula (I) serve as thickeners and produce exceptional viscosity control. Compounds of formula (I): do not depress the foaming of shampoos; may be used to control and maintain the viscosity of personal care products such as shampoos, hair conditioners, hair permanent waves, hair dyes, hair bleaches, hair relaxers, shaving products, skin cleansers, skin creams, skin lotions and soaps; exhibit thickening properties that are comparable or superior to other ethoxylated thickeners; are stable over a broad pH range (i.e., from a pH of less than about 1 to more than about 13, more particularly, from a pH of about 1 to about 13); and can be used in combination with anionic, cationic, amphoteric and nonionic surfactant systems. Solutions comprising thickeners of formula (I) solubilized in water and anionic, cationic, amphoteric, or nonionic surfactants, are substantially clear.

These and other aspects of the invention are disclosed further in the following detailed description.

### DETAILED DESCRIPTION OF THE INVENTION

As used herein, the following terms have the following respective meanings. Other terms that are used to describe the present invention have the same definitions as those generally used by those skilled in the art. Specific examples recited in any definition are not intended to be limiting in any way.

“Alkyl” refers to a fully saturated monovalent hydrocarbon radical (containing 1 to about 24 carbon atoms) and hydrogen which may be a straight chain, branched, or cyclic. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl as well as lauryl, myristyl, palmityl, stearyl and behenyl, among others. The term “alkyl” subsumes the term “alkylene” within context.

“Alkenyl” refers to a branched or unbranched hydrocarbon group typically although not necessarily containing from 2 to about 24 carbon atoms and at least one double bond, such as ethenyl, n-propenyl, isopropenyl, n-butenyl, isobutenyl, octenyl, decenyl, oleyl, linoleyl, linolenyl, erucyl and the like. Generally, although again not necessarily, alkenyl groups herein contain 12 to about 24, preferably 15 to about 24 carbon atoms.

The term “effective amount” is used throughout the specification to describe concentrations or amounts of compounds according to the present invention which are effective in conveying desired traits such as emulsification, clarification, adhesion, melting point modification or solubility to a formulation of a cosmetic, toiletry or personal care product.

The term “personal care product” is used throughout the specification to describe a cosmetic or toiletry product which is preferably used on or in contact with the hair, skin and/or nails and which include effective concentrations of one or more of the compositions according to the present invention. Personal care products include, for example, cosmetics, floating bath oils, after shaves, creams, lotions, deodorants, including stick deodorants, pre-electric shave lotions, after-shave lotions, antiperspirants, shampoos, conditioners and rinses and related products, among others, including skin care products, eye makeups, body shampoos, protective skin formulations, lipsticks, lip glosses, after-bath splashes, presun and sun products, including sunscreens. Virtually any chemical product which comes into contact with the hair or skin and which may include effective amounts or concentrations of one or more of the compositions according to the present invention may be considered a personal care product according to the present invention.

The term “stability” shall mean stable (i.e., has an enduring quality which resists breaking down over time) at a temperature of no greater than about 50° C. for a period of at least about three months, preferably at least about 6 months, more preferably at least about one year or more. The present compositions exhibit stability over a wide range of pH, a surprising characteristic.

Ethoxylated fatty alcohol urethane dimers of formula (I) can be combined in personal care products in accordance with the invention with a variety of surfactant systems. Such surfactants include those that function as detergents to clean the hair. Conventional surfactants such as anionic, cationic and amphoteric surfactants can be used. In shampoo formulations, sodium-based surfactants are sometimes preferred over ammonium-based surfactants. Commercial sources of such surfactants can be found in McCutcheon's EMULSIFIERS AND DETERGENTS, North American Edition, 1984, McCutcheon Division, MC Publishing Company, the complete disclosure of which is hereby incorporated by reference. The amount of surfactant can range from about 1% to about 70% on a weight percentage basis, more typically from about 2% to about 50%. Preferred surfactants include ammonium lauryl sulfate, ammonium laureth sulfate, triethanolammonium lauryl sulfate, triethanolammonium laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauroyl sarcosine, cocoyl sarcosine, monoethanolamine lauryl sulfate, sodium tridecyl benzene sulfonate, sodium dodecyl benzene sulfonate, and cocoamphocarboxyglycinate. Ammonium laureth sulfate and sodium laureth sulfate are particularly preferred.

Ethoxylated fatty alcohol urethane dimers of formula (I) can be combined in personal care products in accordance with the invention with a variety of auxiliary conditioning agents such as quaternary ammonium compounds, amines, amine salts and other cationic polymers. Among the quaternary ammonium compounds which may be used are quaternary ammonium hydroxides, such as methyl triethanol ammonium hydroxide and tetraethyl ammonium hydroxide, among others. Preferred auxiliary conditioning agents include Stearalkonium chloride, Lapyrium chloride, steapyrium chloride, polyquatemium 10, polyquatemium 7, guar hydroxypropyltrimonium chloride or behentrimonium methosulfate. Such auxiliary conditioning agents can be used in an amount of from about 0.1% to about 3.0% on a weight percentage basis, preferably from about 0.5% to about 2.0%.

Ethoxylated fatty alcohol urethane dimers of formula (I) can be combined in personal care products in accordance with the invention with a variety of emollients including, but are not limited to, mineral oil, vegetable oils, cosmetic esters, silicone oils and petrolatum. Other emollients may include cetyl or stearyl alcohol, paraffin or lanolin alcohol. Emollients are generally employed in the formulations of the instant invention in a weight percentage range of from about 5% to about 45%, preferably from about 7.5% to about 40%.

Examples of suitable humectants include, but are not limited to, propylene glycol, hexylene glycol, glycerin and sorbitol. As a general guide, humectants are used in a weight percentage range of from about 1% to about 20%, preferably from about 4% to about 10%.

Ethoxylated fatty alcohol urethane dimers of formula (I) can be combined in personal care products in accordance with the invention with a variety of emulsifying agents. Emulsifiers typically provide dispersion and suspension of the components, and render a creamy and lubricous consistency to the composition. Nonlimiting examples of emulsifying agents suitable for use include alkoxylated alcohols and fatty alcohols, such as stearyl, cetyl and cetearyl alcohols, ethoxylated sorbitan esters, ethoxylated lanolin and derivatives thereof. As a general guide, emulsifiers can be used in amounts of about 1% to about 16% on a weight percentage basis, preferably from about 2% to about 12%, and more preferably from about 8% to about 10%.

In the instant invention, ethoxylated fatty alcohol urethane dimers of formula (I) are included in personal care products/formulations in effective amounts, i.e., amounts which produce an intended effect. The amount of ethoxylated fatty alcohol urethane dimer of formula (I) generally ranges from about 0.5% to about 10% by weight or more of personal care formulations according to the present invention. In preferred embodiments, ethoxylated fatty alcohol urethane dimers of formula (I) are included in amounts ranging from about 0.5% to about 5% by weight. In preferred shampoos and conditioners, ethoxylated fatty alcohol urethane dimers of formula (I) are included in amounts ranging from about 1.0% to about 3% by weight of the formulation.

Ethoxylated fatty alcohol urethane dimers of formula (I) can be combined in personal care products in accordance with the invention with a variety of supplementary thickeners such as natural gums such as tragacanth, xanthan, acacia and locust bean, and synthetic gums such as hydroxypropylcellulose, hydroxyethyl cellulose and hydroxypropyl methylcellulose. Polyvinyl alcohols and Polyvinylpyrrolidone can also be used. Alkanolamides, “super” amides and the glycol or glycerol stearates may also be used.

Ethoxylated fatty alcohol urethane dimers of formula (I) can be combined in personal care products in accordance with the invention with other optional additives including antimicrobial preservatives, antioxidants such as sodium sulfite, chelating agents such as EDTA, suspending agents, fragrances or perfumes, herbal extracts and pH control agents such as citric acid. These additives are usually present in an amount of less than 5% on a weight percentage basis. Sunscreen agents maybe added in accordance with the FDA monograph regulations. In a shampoo, an antidandruff component, e.g., selenium sulfide, may also be included at an effective level.

Properties and characteristics of the ethoxylated fatty alcohol urethane dimers used in the present invention that make them especially useful as thickeners in personal care, cosmetic, and toiletry application include the following: extremely low order of toxicity and irritation; low color; excellent compatibility in cosmetic formulations; solubility with amides, sulfates, sulfonates sulfosuccinates, and sulfobetaines; nonrancidification; coupling characteristics; solubility in water, glycols and lower molecular weight alcohols; and excellent viscosity stability at extremes of pH.

As described, compounds of formula (I) can be made by the reaction in the presence of heat and either an amine or tin catalyst such as stannous octanoate of an ethoxylated fatty alcohol with a diisocyanate, most preferably isophorone diisocyanate, in an approximately 2:1 molar ratio of ethoxylated fatty alcohol to isophorone diisocyanate, at a temperature of between about 80° C. to about 120° C., at approximately atmospheric pressure, and in accordance with the following reaction scheme: 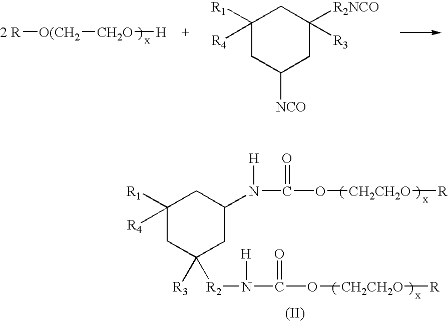
where x is a whole number from about 50 to 120; n is a whole number from 50 to 120, preferably about 70 to 100, more preferably about 65-70, and most preferably about 70;
R is a C₁₂-C₂₄ alkyl or alkenyl group; and
R₁, R₂, R₃ and R₄ are the same or different and are a C, to C₇ alkyl or alkenyl group.

As the number of carbon atoms in the diisocyanate decreases, the number of carbon atoms in the monohydric alcohol may be increased accordingly to maintain similar chemical characteristics, provided the degree of ethoxylation of the ethoxylated fatty alcohol urethane dimers of formula (II) is maintained in the range specified hereinafter. In general, as the molecular weight of the ethoxylated fatty alcohol urethane dimers of formulas (I) or (II) increases as a consequence of the contribution from the non-ethoxylated portion of the molecule, the thickening properties of such compounds will increase. Conversely, if the ethoxylated portion of the molecule increases, the water solubility of the molecule may increase, resulting in a reduced thickening characteristic. Also, in general, as branching of the ethoxylated fatty alcohol urethane dimers of formula (I) or (II) increases, thickening decreases.

Theoretically, the degree of ethoxylation can be any that is desired, but for purposes of building viscosity in cosmetic products such as skin creams and lotions and hair care products such as shampoos, conditioners and relaxers, fatty alcohols having from about 75 to about 150 moles of ethoxylation, more preferably from about 75 to about 100 moles of ethoxylation, are preferred. Urethane dimers of fatty alcohol ethoxylates with from about 100 moles of ethoxylation to 150 moles will typically make a clear solution in water with a viscosity of between 10,000 and 15,000 cps at 5% concentration.

Dimers made from fatty alcohols with lower degrees of ethoxylation (e.g., about 75 moles of ethoxylation) produce hazy/cloudy solutions in water and precipitate a water insoluble, sticky gel. However, when these poorly water soluble dimers are solubilized with an added surfactant, the resulting solution can exhibit a significantly higher viscosity as compared to a more ethoxylated, water soluble dimer.

Preferred fatty alcohols useful in making urethane dimers of fatty alcohol ethoxylates range from myristyl to behenyl alcohol (C₁₄ to C₂₂) and are more preferably either cetyl or stearyl alcohols (C₁₆ and C₁₈) and mixtures thereof. Dimers made with a 75 mole ethoxylate (or, for example, 100 mole ethoxylate) of a fatty alcohol are marketed under the tradename Dermothix 75 (or Dermothix 100). These materials are somewhat comparable in performance to the thickener Crothix (Croda, Inc.) which has similar solubility characteristics. Direct viscosity measurements of Dermothix 75 and Crothix are not possible, as the compositions are not sufficiently water soluble (See Examples 1 & 2 hereinafter). However, these thickeners can be solubilized by the addition of surfactants to make clear, viscous fluids.

For example, a 3% w/w concentration of the 75 mole ethoxylated stearyl alcohol dimer in 3% active sodium laureth sulfate produces a clear solution with a viscosity of 24,000 cps (See Example 5 hereinbelow). By comparison, a 3% solution of Crothix in 3% active SLES2 produces a viscosity of 1,400 cps (See Example 6 hereinafter). Thus, the addition of a solubilizing surfactant helps to dissolve the urethane dimer of fatty alcohol ethoxylate, allowing it to function as desired. Examples 3, 4, 7 and 8 illustrate the thickening effects of the more water soluble thickeners Dermothix 100 and PEG 6000 Distearate.

These and other aspects of the invention are described further in the following examples, which are illustrative and in no way limiting.

### EXAMPLE 1

Materials and Methods

In performing the following syntheses and preparing the following final formulations, the reagents which are used are indicated in the specific examples. Solvents, where used, are preferably distilled prior to use. Sources of other materials are indicated in the appropriate experimental section. In most instances, although not in every instance, trademarked materials are available from Alzo International, Inc., Sayreville, N.J. Other materials are readily available from other chemical manufacturers and suppliers.

Shampoo formulations 1-8 were formulated using the ingredients disclosed in Tables 1-2 and viscosity of the formulations was measured using a Brookfield RVT viscometer. Viscosity measurements for formulations 1-8 are listed in Table 3. Formulations 3 and 7 contained the ethoxylated fatty alcohol urethane dimer Dermothix 100 as a thickener and evidenced exceptional viscosity and clarity.

TABLE 1

| | 1 | 2 | 3 | 4 |
|---|---|---|---|---|
| Ingredients | % | % | % | % |
| Dermothix 75 | 5.0 | — | — | — |
| Crothix | — | 5.0 | — | — |
| Dermothix 100 | — | — | 5.0 | — |
| PEG 6000 Distearate | — | — | — | 5.0 |
| Water | 94.0 | 94.0 | 94.0 | 94.0 |
| Phenobact (antimicrobial) | 1.0 | 1.0 | 1.0 | 1.0 |
| | 100.0 | 100.0 | 100.0 | 100.0 |

TABLE 2

| | 5 | 6 | 7 | 8 |
|---|---|---|---|---|
| Ingredients | % | % | % | % |
| Dermothix 75 | 3.0 | — | — | — |
| Crothix | — | 3.0 | — | — |
| Dermothix 100 | — | — | 3.0 | — |
| PEG 6000 Distearate | — | — | — | 3.0 |
| Sodium Laureth(2) Sulfate (30% active) | 10.0 | 10.0 | 10.0 | 10.0 |
| Water | 86.0 | 86.0 | 86.0 | 86.0 |
| Phenobact | 1.0 | 1.0 | 1.0 | 1.0 |
| | 100.0 | 100.0 | 100.0 | 100.0 |

TABLE 3
Viscosity Results

| Formulation | Viscosity (cps) | comments |
|---|---|---|
| 1 | — | Not soluble |
| 2 | — | Not soluble |
| 3 | 12,500 | Clear liquid |
| 4 | 1,600 | Clear liquid |
| 5 | 24,000 | Clear liquid |
| 6 | 1,400 | Clear liquid |
| 7 | 12,500 | Clear liquid |
| 8 | 25,500 | Clear liquid |

### EXAMPLE 2

The dimer urethane ethoxylates are also determined to function as thickeners when used in combination with cationic surfactants in hair conditioner formulations 9, 10, 11, 12 and 13 Table 4. The PEG 6000 Distearate was especially effective as a thickener, possibly due to complexation between the stearalkonium chloride and free stearic acid that is residual from the esterification of the PEG 6000. Viscosity measurements for formulations 9-13 were determined as in Example 1 and are set forth in Table 5.

TABLE 4

| | 9 | 10 | 11 | 12 | 13 |
|---|---|---|---|---|---|
| Ingredients | % | % | % | % | % |
| Dermothix 75 | 3.0 | — | — | — | — |
| Crothix | — | 3.0 | — | — | — |
| Dermothix 100 | — | — | 3.0 | — | — |
| PEG 6000 Distearate | — | — | — | 3.0 | — |
| Glucamate DOE 120 | — | — | — | — | 3.0 |
| Stearalkonium Chloride | 3.0 | 3.0 | 3.0 | 3.0 | 3.0 |
| Water | 93.0 | 93.0 | 93.0 | 93.0 | 93.0 |
| Phenobact | 1.0 | 1.0 | 1.0 | 1.0 | 1.0 |
| | 100.0 | 100.0 | 100.0 | 100.0 | 100.0 |

TABLE 5
Viscosity Results

| Example# | Viscosity (cps) | comments* |
|---|---|---|
| 9 | 28,800 | Clear Liquid |
| 10 | 4,600 | Clear liquid |
| 11 | 65,600 | Clear liquid |
| 12 | 142,000 | Hazy/cloudy liquid |
| 13 | 5 | Clear liquid |

*Samples were stored at 50° C. for 24 hours, then at RT (22° C.) for 6 hrs. Sample temperature was 22° C. prior to viscosity measurement. Subsequently (overnight) the samples showed precipitates and crystallization.

### EXAMPLE 3

The dimer urethane ethoxylates were also determined to function as thickeners when used in combination with amphoteric surfactants in shampoo formulations 14, 15, 16, 17, and in Table 6. The amphoteric surfactant used to make these formulations was Foamtaine CAB from Alzo International, Inc., which has the INCI name Cocamidopropyl Betaine. Viscosity values for formulations 14-18 were determined as in Example 1 and are listed in Table 7. The solubility of the thickener in the surfactant solution was determined to be critical to achieving a functional viscosity in the final formula. The more soluble the thickener is in the solution, the less viscosity it produces.

TABLE 6

| | 14 | 15 | 16 | 17 | 18 |
|---|---|---|---|---|---|
| Ingredients | % | % | % | % | % |
| Dermothix 75 | 3.0 | — | — | — | — |
| Crothix | — | 3.0 | — | — | — |
| Dermothix 100 | — | — | 3.0 | — | — |
| PEG 6000 Distearate | — | — | — | 3.0 | — |
| Glucamate DOE 120 | — | — | — | — | 3.0 |
| Foamtaine CAB (30% active) | 10.0 | 10.0 | 10.0 | 10.0 | 10.0 |
| Water | 86.0 | 86.0 | 86.0 | 86.0 | 86.0 |
| Phenobact | 1.0 | 1.0 | 1.0 | 1.0 | 1.0 |
| | 100.0 | 100.0 | 100.0 | 100.0 | 100.0 |

TABLE 7
Viscosity Results

| Example# | Viscosity (cps) | comments |
|---|---|---|
| 14 | — | Cloudy 2 phase gel - not soluble |
| 15 | — | Cloudy 2 phase gel - not soluble |
| 16 | 17,800 | Clear liquid |
| 17 | 5,400 | Clear liquid |
| 18 | 12.5 | Clear liquid |

### EXAMPLE 4

Solutions of ethoxylated nonionic surfactants can also be thickened using the dimer urethane ethoxylates, as evidenced by shampoo formulations 19-30 of Tables 8-10. The ethoxylated nonionic surfactant used in these examples was Hetoxol CA-20 (INCI name Ceteth-20)(Global Seven, Inc.). Glucamate DOE 120 was not used in these studies as it had shown poor viscosity building effects in preliminary evaluations. Viscosity values for formulations 19-30 were determined as in Example 1 and are listed in Table 11.

TABLE 8

| | 19 | 20 | 21 | 22 |
|---|---|---|---|---|
| Ingredients | % | % | % | % |
| Dermothix 75 | 1.0 | — | — | — |
| Crothix | — | 1.0 | — | — |
| Dermothix 100 | — | — | 1.0 | — |
| PEG 6000 Distearate | — | — | — | 1.0 |
| Ceteth-20 | 5.0 | 5.0 | 5.0 | 5.0 |
| Water | 93.0 | 93.0 | 93.0 | 93.0 |
| Phenobact | 1.0 | 1.0 | 1.0 | 1.0 |
| | 100.0 | 100.0 | 100.0 | 100.0 |

TABLE 9

| | 23 | 24 | 25 | 26 |
|---|---|---|---|---|
| Ingredients | % | % | % | % |
| Dermothix 75 | 3.0 | — | — | — |
| Crothix | — | 3.0 | — | — |
| Dermothix 100 | — | — | 3.0 | — |
| PEG 6000 Distearate | — | — | — | 3.0 |
| Ceteth-20 | 5.0 | 5.0 | 5.0 | 5.0 |
| Water | 91.0 | 91.0 | 91.0 | 91.0 |
| Phenobact | 1.0 | 1.0 | 1.0 | 1.0 |
| | 100.0 | 100.0 | 100.0 | 100.0 |

TABLE 10

| | 27 | 28 | 29 | 30 |
|---|---|---|---|---|
| Ingredients | % | % | % | % |
| Dermothix 75 | 5.0 | — | — | — |
| Crothix | — | 5.0 | — | — |
| Dermothix 100 | — | — | 5.0 | — |
| PEG 6000 Distearate | — | — | — | 5.0 |
| Ceteth-20 | 5.0 | 5.0 | 5.0 | 5.0 |
| Water | 89.0 | 89.0 | 89.0 | 89.0 |
| Phenobact | 1.0 | 1.0 | 1.0 | 1.0 |
| | 100.0 | 100.0 | 100.0 | 100.0 |

TABLE 11
Viscosity Results

| Example# | Viscosity (cps) | comments |
|---|---|---|
| 19 | 100 | Clear liquid -1% Thickener |
| 20 | 12.5 | Clear liquid -1% Thickener |
| 21 | 12.5 | Clear liquid -1% Thickener |
| 22 | 12.5 | Hazy liquid -1% Thickener |
| 23 | 19,500 | Clear liquid -3% Thickener |
| 24 | 3750 | Clear liquid -3% Thickener |
| 25 | 20,000 | Clear liquid -3% Thickener |
| 26 | 2,750 | Hazy/cloudy liquid -3% Thickener |
| 27 | 125,000 | Clear liquid -5% Thickener |
| 28 | 27,500 | Clear liquid -5% Thickener |
| 29 | 85,000 | Clear liquid -5% Thickener |
| 30 | 14,250 | Hazy/cloudy liquid -5% Thickener |

As evidenced by the viscosity values of Table 11, the composition and amount of the thickener added directly affects the viscosity of the surfactant solution. The solubility of the thickener in the surfactant solution is critical to achieving a functional viscosity in the final formula. The more soluble the thickener is in the solution, the less viscosity it produces. The hazy/cloudiness seen in the PEG 6000 Distearate solutions is attributed to the presence of free fatty acids that were not completely solubilized by the nonionic.

### EXAMPLE 5

The formulations prepared in this Example were used to test the thickeners at pH extremes (formulations 31 through 38, Tables 12 and 13). Viscosity values for formulations 31-38 were determined as in Example 1 and are listed in Table 14. The results of the experiments in this Example show that the urethane dimer thickeners are effective and stable at both high and low pH values, while the ester type thickeners are not useful under such conditions.

TABLE 12

| | 31 | 32 | 33 | 34 |
|---|---|---|---|---|
| Ingredients | % | % | % | % |
| Dermothix 75 | 3.0 | — | — | — |
| Crothix | — | 3.0 | — | — |
| Dermothix 100 | — | — | 3.0 | — |
| PEG 6000 Distearate | — | — | — | 3.0 |
| Ceteth-20 | 5.0 | 5.0 | 5.0 | 5.0 |
| Hydrochloric acid (37%) | 1.0 | 1.0 | 1.0 | 1.0 |
| Water | 90.0 | 90.0 | 90.0 | 90.0 |
| Phenobact | 1.0 | 1.0 | 1.0 | 1.0 |
| | 100.0 | 100.0 | 100.0 | 100.0 |
| pH | 1.13 | 1.15 | 1.18 | 1.15 |

TABLE 13

| | 35 | 36 | 37 | 38 |
|---|---|---|---|---|
| Ingredients | % | % | % | % |
| Dermothix 75 | 3.0 | — | — | — |
| Crothix | — | 3.0 | — | — |
| Dermothix 100 | — | — | 3.0 | — |
| PEG 6000 Distearate | — | — | — | 3.0 |
| Ceteth-20 | 5.0 | 5.0 | 5.0 | 5.0 |
| Sodium Hydroxide (50%) | 0.8 | 0.8 | 0.8 | 0.8 |
| Water | 90.2 | 90.2 | 90.2 | 90.2 |
| Phenobact | 1.0 | 1.0 | 1.0 | 1.0 |
| | 100.0 | 100.0 | 100.0 | 100.0 |
| pH | 12.69 | 12.70 | 12.67 | 12.71 |

TABLE 14
Viscosity Results:

| | 24 hr. | After 5 days | After 2 weeks |
|---|---|---|---|
| Example# | Viscosity (cps) | Viscosity (cps) | Viscosity(cps) |
| 31 | 15,250 | 18,000 | 15,000 |
| 32 | 2,250 | 47.5 | <10 |
| 33 | 17,000 | 20,200 | 17,000 |
| 34 | 1,400 | 10 | <10 |
| 35 | 12,000 | 12,500 | 9,000 |
| 36 | 5 | 5 | 5 |
| 37 | 14,750 | 16,500 | 10,250 |
| 38 | 5 | 5 | 5 |

### EXAMPLE 6

Shampoo formulations were made with various thickeners in accordance with the invention and were determined to have the compositions and properties set forth in Tables 15-17. Viscosity values were determined as in the experiment of Example 1.

TABLE 15

| | 39 | 40 | 41 | 42 |
|---|---|---|---|---|
| Ingredients | % | % | % | % |
| Dermothix 75 | 1.0 | — | — | — |
| Crothix | — | 1.0 | — | — |
| Dermothix 100 | — | — | 1.0 | — |
| PEG 6000 Distearate | — | — | — | 1.0 |
| Sodium Laureth(2) Sulfate | 30.0 | 30.0 | 30.0 | 30.0 |
| Foamtaine CAB | 10.0 | 10.0 | 10.0 | 10.0 |
| Water | 58.0 | 58.0 | 58.0 | 58.0 |
| Phenobact | 1.0 | 1.0 | 1.0 | 1.0 |
| | 100.0 | 100.0 | 100.0 | 100.0 |

TABLE 16

| | 43 | 44 | 45 | 46 | 47 |
|---|---|---|---|---|---|
| Ingredients | % | % | % | % | % |
| Dermothix 75 | 2.0 | — | — | — | — |
| Crothix | — | 2.0 | — | — | — |
| Dermothix 100 | — | — | 2.0 | — | — |
| PEG 6000 Distearate | — | — | — | 2.0 | — |
| Sodium Laureth(2) Sulfate | 30.0 | 30.0 | 30.0 | 30.0 | 30.0 |
| Foamtaine CAB | 10.0 | 10.0 | 10.0 | 10.0 | 10.0 |
| Water | 57.0 | 57.0 | 57.0 | 57.0 | 59.0 |
| Phenobact | 1.0 | 1.0 | 1.0 | 1.0 | 1.0 |
| | 100.0 | 100.0 | 100.0 | 100.0 | 100.0 |

TABLE 17
Viscosity Results:

| | | Initial/Final Foam | |
|---|---|---|---|
| Formulation | Viscosity (cps) | Volume (mls)* | Comments |
| 39 | 15,000 | 500/450 | Clear liquid |
| 40 | 22,000 | 410/360 | Clear liquid |
| 41 | 2,200 | 375/325 | Clear liquid |
| 42 | 7,000 | 410/360 | Clear liquid |
| 43 | 85,000 | 420/370 | Clear liquid |
| 44 | 82,500 | 450/410 | Clear liquid |
| 45 | 27,500 | 390/340 | Clear liquid |
| 46 | 50,000 | 410/370 | Clear liquid |
| 47 | 25 | 410/360 | Clear liquid |

The results of the experiments of this Example show that in a typical shampoo formula, the thickeners are all effective at increasing viscosity, although to a varying degree, and they do not have a deleterious effect upon the foam volumes generated by the shampoo.

The foam test used was a modified “rotating cylinder” type. A 1 liter, graduated, stopper top, volumetric cylinder was used with 100 ml of 1% w/w, as is, shampoo solution at room temperature. An axis was fitted at the 600 ml mark, and the cylinder was rotated, by hand, about this axis for 20 half revolutions—first in one direction, followed by return to vertical. The recorded initial foam volume was the total measured foam volume plus liquid volume measured immediately when the 20 half revolutions are completed, minus the volume of liquid that drains from the foam at the point where a stable, observable liquid/foam interface first forms. The final foam volume value was the total foam volume measured after five minutes minus the total liquid that has drained out of the foam during that time. These values reflect the stability of the foam.

### EXAMPLE 7

Baby shampoo formulations 48-52 were made in accordance with the invention using the ingredients specified in Table 18. Viscosity values for the formulations were determined as in Example 1 and are listed in Table 19.

TABLE 18

| | 48 | 49 | 50 | 51 | 52 |
|---|---|---|---|---|---|
| Ingredients | % | % | % | % | % |
| Dermothix 75 | 2.0 | — | — | — | — |
| Crothix | — | 2.0 | — | — | — |
| Dermothix 100 | — | — | 2.0 | — | — |
| PEG 6000 Distearate | — | — | — | 2.0 | — |
| Sodium Laureth(2) Sulfate | 15.0 | 15.0 | 15.0 | 15.0 | 15.0 |
| Miranol C2 M conc. NP | 15.0 | 15.0 | 15.0 | 15.0 | 15.0 |
| Polysorbate 20 | 15.0 | 15.0 | 15.0 | 15.0 | 15.0 |
| Water | 52.0 | 52.0 | 52.0 | 52.0 | 54.0 |
| Phenobact | 1.0 | 1.0 | 1.0 | 1.0 | 1.0 |
| | 100.0 | 100.0 | 100.0 | 100.0 | 100.0 |

TABLE 19
Viscosity Results:

| | | Initial/Final Foam | |
|---|---|---|---|
| Example# | Viscosity (cps) | Volume (mls)* | Comments |
| 48 | 350 | 170/150 | Clear liquid |
| 49 | 525 | 230/200 | Clear liquid |
| 50 | 50 | 230/200 | Clear liquid |
| 51 | 50 | 160/140 | Clear liquid |
| 52 | 12.5 | 170/150 | Clear liquid |

The formulations illustrated in this Example show that in a baby shampoo formula, the thickeners are effective at increasing the viscosity, although to varying degrees, and do not have a deleterious effect upon the foam volumes generated by the shampoo.

### EXAMPLE 8

Emulsion formulations 53-57 (Table 20) were prepared in accordance with the invention. These emulsion formulations were thickened with ethoxylated fatty alcohol urethane dimers. Viscosity values for the formulations were determined using a Brookfield LVT viscometer equipped with a heliopath stand and a T-F spindle and are listed in Table 21.

TABLE 20

| | 53 | 54 | 55 | 56 | 57 |
|---|---|---|---|---|---|
| Ingredients | % | % | % | % | % |
| Dermothix 75 | 1.0 | — | — | — | — |
| Crothix | — | 1.0 | — | — | — |
| Dermothix 100 | — | — | 1.0 | — | — |
| PEG 6000 Distearate | — | — | — | 1.0 | — |
| Ceteareth 14 | 1.0 | 1.0 | 1.0 | 1.0 | 1.0 |
| Cetyl Alcohol | 2.0 | 2.0 | 2.0 | 2.0 | 2.0 |
| Stearyl Alcohol | 2.0 | 2.0 | 2.0 | 2.0 | 2.0 |
| Mineral Oil | 15.0 | 15.0 | 15.0 | 15.0 | 15.0 |
| Water | 76.0 | 76.0 | 76.0 | 76.0 | 77.0 |
| Glycerin | 2.0 | 2.0 | 2.0 | 2.0 | 2.0 |
| Phenobact | 1.0 | 1.0 | 1.0 | 1.0 | 1.0 |
| | 100.0 | 100.0 | 100.0 | 100.0 | 100.0 |

TABLE 21
Viscosity Results:

| Formulation | Viscosity (cps) | comments |
|---|---|---|
| 53 | 399,000 | Thick cream |
| 54 | 2,028,000 | Thick cream |
| 55 | 1,560,000 | Thick cream |
| 56 | 1,061,000 | Thick cream |
| 57 | 218,000 | Very thick lotion (some flow) |

The results of the experiments of this Example showed that the thickeners are effective, to differing degrees, at increasing viscosity in the illustrated emulsions. Dermothix 100 was determined to be more effective as an emulsion thickener than Dermothix 75. These results are in contrast to those of the shampoo and single surfactant studies (e.g. Example 1) in which Dermothix 75 was determined to be consistently better than Dermothix 100 at increasing viscosity.

It is to be understood by those skilled in the art that the foregoing descriptions and examples are illustrative of practicing the present invention, but are in no way limiting. Variations of the details presented herein may be made without departing from the spirit and scope of the present invention as defined by the following claims.

1. A personal care product comprising a substantially solubilized fatty ethoxylated dimeric urethane of the formula: 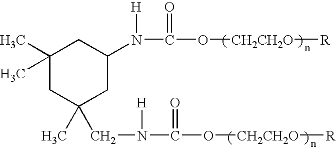
where n is a whole number from about 50 to 120;
R is a C₁₂-C₂₄ alkyl or alkenyl group; and
wherein the fatty ethoxylated dimeric urethane comprises about 0.5% to about 10% of the personal health care product on a weight percentage basis and the viscosity of the personal health care product ranges from about 500 to about 500,000 cps, preferably about 5,000 to about 150,000 cps.

2. The personal care product of claim 1, wherein the personal care product is a shampoo, n is a whole number from about 70 to 100, and the fatty ethoxylated dimeric urethane comprises about 1% to about 5% of the personal health care product on a weight percentage basis.

3. The personal care product of claim 1, wherein the personal care product is an emulsion, n is a whole number from about 70 to 100, and the fatty ethoxylated dimeric urethane comprises about 1% to about 5% of the personal health care product on a weight percentage basis.

4. The personal care product of claim 1, wherein the personal care product comprises one or more anionic, cationic, amphoteric or nonionic surfactants.

5. The personal care product of claim 1, wherein the fatty ethoxylated dimeric urethane is made by reacting an ethoxylated fatty alcohol with isophorone diisocyanate in an approximately 2:1 molar ratio of ethoxylated fatty alcohol to isophorone diisocyanate, wherein the ethoxylated fatty alcohol has the formula
2 R—O(CH₂—CH₂O)ₓ—H
where x is a whole number from about 50 to 120 and R is a C₁₂-C₂₄ alkyl or alkenyl group, and wherein the ethoxylated fatty alcohol and isophorone diisocyanate react in the presence of heat and either an amine or tin catalyst, at a temperature of between about 80° C. to about 120° C., and at approximately atmospheric pressure.

6. The personal care product of claim 5, wherein the fatty ethoxylated dimeric urethane is solubilized by the addition of one or more surfactants.

7. The personal care product of claim 5, wherein the fatty ethoxylated dimeric urethane contains from about 130 to about 200 moles of ethylene oxide.

8. The personal care product of claim 5, wherein the ethoxylated fatty alcohol is a C₁₄ to C₂₂ alcohol.

9. The personal health care product of claim 5, wherein the fatty ethoxylated dimeric urethane is Dermothix 75 or Dermothix 100.

10. A method comprising increasing the viscosity of a personal care product by solubilizing into the personal health care product a fatty ethoxylated dimeric urethane of the formula: 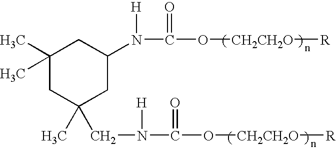
where n is a whole number from about 50 to 120;
R is a C₁₅-C₂₄ alkyl or alkenyl group; and
wherein the fatty ethoxylated dimeric urethane comprises about 0.5% to about 10% of the personal health care product on a weight percentage basis and the viscosity of the personal health care product ranges from about 500 to about 500,000 , preferably about 5,000 to 150,000 cps.

11. A personal care product comprising a fatty ethoxylated dimeric urethane made by reacting an ethoxylated fatty alcohol with a diisocyanate in an approximately 2:1 molar ratio of ethoxylated fatty alcohol to diisocyanate, wherein: the ethoxylated fatty alcohol has the formula
2 R—O(CH₂—CH₂O)ₓ—H
the diisocyanate has the formula 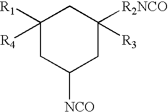
wherein x is a whole number from about 50 to 120;
R is a C₁₅-C₂₄ alkyl or alkenyl group; and
R₁, R₂, R₃ and R₄ are the same or different and are a Cl to C₇ alkyl or alkenyl group.

12. The personal care product of claim 1, wherein the fatty ethoxylated dimeric urethane contains from about 75 to about 150 moles of ethoxylation.

13. The personal care product of claim 12, wherein the product viscosity is between about 10,000 and 15,000 cps.

14. The personal care product of claim 12, wherein the product viscosity is between about 10,000 and 15,000 cps and the concentration of the fatty ethoxylated dimeric urethane is approximately 5% to 10% by weight of the product.

15. The personal care product of claim 12, wherein the product comprises a surfactant that solubilizes the fatty ethoxylated dimeric urethane.

16. The personal care product of claim 1 which exhibits stability at a pH ranging from about 1.0 to about 13.0.

17. The personal care product of claim 9 which exhibits stability at a pH ranging from about 1.0 to about 13.0.

18. The personal care product of claim 11 which exhibits stability at a pH ranging from about 1.0 to about 13.0.

19. The personal care product of claim 11 wherein said diisocyanate is isophorone diisocyanate.

20. A personal care product comprising a substantially solubilized fatty ethoxylated dimeric urethane of the formula: 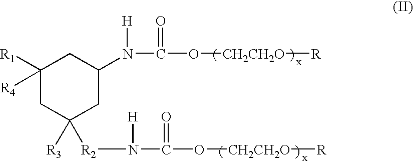
where x is a whole number from about 50 to 120; n is a whole number from 50 to 120, preferably about 70 to 100, more preferably about 65-70, and most preferably about 70;
R is a C₁₂-C₂₄ alkyl or alkenyl group; and
R₁, R₂, R₃ and R₄ are the same or different and are a C, to C₇ alkyl or alkenyl group, wherein the fatty ethoxylated dimeric urethane comprises about 0.5% to about 10% of the personal health care product on a weight percentage basis and the viscosity of the personal health care product ranges from about 500 to about 500,000 cps, preferably about 5,000 to about 150,000 cps.
